(19) United States
(12) Patent Application Publication
(10) Pub. No.: US 2016/0310522 A1
(43) Pub. Date: Oct. 27, 2016

(54) METHOD FOR PRODUCING COMPOSITE GEL BY CROSS-LINKING HYALURONIC ACID AND HYDROXYLPROPYL METHYLCELLULOSE

(71) Applicant: AiMeike Technology Deveolpment., LTD, BEIJING (CN)

(72) Inventors: Jun Jian, Beijing (CN); Ruizhi Li, Beijing (CN)

(73) Assignee: AiMeike Technology Deveolpment., LTD

(21) Appl. No.: 15/202,664

(22) Filed: Jul. 6, 2016

Related U.S. Application Data
(63) Continuation-in-part of application No. 13/844,842, filed on Mar. 16, 2013, now abandoned.
Application No. 13/844,842 is a continuation-in-part of application No. PCT/CN2011/084096, filed on Dec. 16, 2011.

(30) Foreign Application Priority Data
Apr. 26, 2011 (CN) 201110104213.5
Sep. 28, 2011 (CN) 201110289906.6
Dec. 1, 2011 (CN) 201110392570.6
Dec. 1, 2011 (CN) 201110392621.5
Dec. 1, 2011 (CN) 201110392623.4
Dec. 1, 2011 (CN) 201110392624.9

(51) Int. Cl. A61K 31/738

(52) U.S. Cl. CPC A61K 31/738

(57) ABSTRACT

A method for covalently cross-linking hyaluronic acid (HA) and hydroxypropyl methylcellulose (HPMC) by a diepoxide crosslinking agent. The method includes the following steps: a) mixing HA and HPMC in water; b) adding an alkali as a catalyst and a diepoxide as a crosslinking agent; c) neutralizing with hydrochloric acid and dehydrating with ethanol and acetone; and d) drying in vacuum and redissolving in water to obtain an HA-HPMC composite gel.

### CROSS-REFERENCE TO RELATED APPLICATIONS

This application is a continuation-in-part of application Ser. No. 13/844,842 filed Mar. 16, 2013, which is a continuation-in-part of International Patent Application No. PCT/CN2011/084096 with an international filing date of Dec. 16, 2011, designating the United States, now pending, and further claims priority benefits to Chinese Patent Application No. 201110104213.5 filed Apr. 26, 2011, to Chinese Patent Application No. 201110289906.6 filed Sep. 28, 2011, to Chinese Patent Application No, 201110392570.6 filed Dec. 1, 2011, to Chinese Patent Application No. 201110392621.5 filed Dec. 1, 2011, to Chinese Patent Application No. 201110392623.4 filed Dec. 1, 2011, and to Chinese Patent Application No. 201110392624.9 filed Dec. 1, 2011. The contents of all of the aforementioned applications, including any intervening amendments thereto, are incorporated herein by reference.

### BACKGROUND OF THE INVENTION

1. Field of the Invention

The invention relates to a crosslinked gel composition of hyaluronic acid (HA) and hydroxypropyl methylcellulose (HPMC) and its preparation methods, both butanediol diglycidyl ether (BDDE) and/or 1,2,7,8-diepoxyoctane (DEO) were used as crosslinking agents. The advantages of the invention are the crosslinking reaction was carried out at mild condition, the high utilization percentage of the crosslinking agents and low residue, the high thermostability and biocompatibility.

2. Description of the Related Art:

Hyaluronic acid is a member of a class of polymers known as glycosaminoglycans. It is a naturally occurring linear polysaccharide composed of alternating N-acetyl-D-glucosamine and D-glucuronic acid monosaccharide units linked via.beta.-1,4-bonds, with the disaccharide units linked via.beta.-1,3-glycoside bonds. Hyaluronic acid usually occurs as salts such as sodium and potassium hyaluronates, The sodium salt has a molecular formula of (C.sub14H.sub.20NNaO.sub.11).sub.n where n can vary according to the source, isolation procedure and method of determination, The molecular weight generally falls between about 6.times.10.sup,4 and about 1.4.times.10.sup.7 Daltons. The term “hyaluronan” (HA) usually refers to both hyaluronic acid and its salts. HA is non-immunogenic and non-toxic. When implanted or injected into a living body, however, HA typically is degraded by oxidation and by enzymes such hyaluronidase. Because HA is a water-soluble polymer and is degraded and eliminated rapidly in vivo, the potential applications for HA in biomedical purposes have been somewhat limited.

Hydroxypropyl methylcellulose, also referred to as “HPMC”, is a non-calorific and safety pharmaceutical excipient. HPMC was widely used as tablet, sustained release preparation, controlled release preparation, ophthalmic drug delivery system, suspension, hydrogel and ointments etc 7 dosage forms.

Methods for preparing commercially available hyaluronan are well known. Also known are various methods for coupling HA and cross-linking HA to reduce the water solubility and diffusibility of HA, and to increase the viscosity of HA. See, for example, U.S. Pat. Nos. 5,356,883 and 6,013,679, the entire teachings of which are incorporated herein by reference. Further, many forms of HA have been employed, e.g., as surgical aids to prevent post operative adhesions of tissues, as adjuncts to synovial fluid in joints, as fluid replacement and/or surgical aids in ophthalmic surgery, as a scaffold for tissue engineering in vitro or guided tissue regeneration or augmentation in vivo, and the like.

At present, residence time of the fashion-market and injection-level HA gels under skin is about one year. Though hydrolysis in vitro via hyaluronidase, the HA gels were degraded completely in two hours.

When 1,2,7,8-diepoxyoctane (DEO) as cross-linking agents, DEO has low solubility under water (less than 1%) because of its hydrophobicity. The characteristics of DEO lead to its crosslinking reactivity less than butanediol diglycidyl ether (BDDE) which can dissolve in water. General experiments are used DEO to prepare low degree of crosslinking gels (<10%). If the amount of crosslinking agent increased more than two times, the utilization percentage of DEO will be very low (<20%) in crosslinking reaction. And the composite gel of high crosslinking degree could be hardly prepared. According to Material Safety Data Sheets (MSDS), DEO has irritant even toxicity of skin. So DEO must be eliminated to the safe content range after crosslinking reaction to avoid residual crosslinking agent on the adverse effects of skin.

### SUMMARY OF THE INVENTION

In view of the above described problems, it is one objective of the invention to provide a composite gel with crosslinking HA and HPMC and a method for making and using the HA and HPMC composition that is effective for tissue augmentation. The crosslinking reaction of the invention is applicable at mild condition, has high utilization percentage of the crosslinking agents and low residue; the composite gel of the invention has high thermostability and biocompatibility.

A HA and HPMC composition comprises crosslinked, water-insoluble, hydrated HA and HPMC gel particles.

A method for preparing the HA and HPMC composition (Method No. 1) comprises: forming water-insoluble and dehydrated crosslinked HA-HPMC particles with hydrophilic crosslinking agents such as butanediol diglycidyl ether (BDDE) via etherification in strong alkalis condition; separating the water-insoluble and dehydrated particles with acetone by average diameter; selecting a subset of particles by average diameter; washing the subset of dehydrated particles with ethanol and acetone successfully; and drying the particles to obtain the HA-HPMC composition.

Another method for preparing the crosslinkod HA-HPMC composition (Method No. 2) comprises: forming water-insoluble and dehydrated crosslinked HA-HPMC particles with hydrophobic crosslinking agents such as 1,2,7,8-diepoxyoctane (DEO) with quaternary ammonium hydroxide as catalyst via etherification in strong alkalis condition firstly and esterification in weak acid condition followed; separating the water-insoluble and dehydrated particles with acetone by average diameter; selecting a subset of particles by average diameter; washing the subset of dehydrated particles with ethanol and acetone successfully; and drying the particles to obtain the HA-HPMC composition.

The specific steps of the method No. 1 comprise:
1) dissolving HA and HPMC in water;
2) adding NaOH as the catalyst, DEO and/or BDDE as the crosslinking agents, reacting for 24-36 h a temperature of 20-30° C. and a pH of 12-14; a mass ratio of BDDE to HA and HPMC is 1:100-3:1 and
3) neutralizing with hydrochloric acid to a pH of 6.5-7.5, electing a subset of particles by an average diameter, washing the subset of dehydrated particles with ethanol and acetone successfully; drying the particles, dissolving with phosphate buffer to obtain a mix solution at a pH of 6.9-7.6.

The specific steps of the method No. 2 comprise:
1) dissolving HPMC, HA and Quaternary Ammonium Hydroxide (QAH) such as tetrabutyl ammonium hydroxide (TBAH) or trimethyloctyl ammonium hydroxide (TMOAH) in watex, contmlling a mass ratio of the HA to HPMC being 100:1-1:1, a mass fraction of QAH being 0.5-30%, a temperature of 20-30° C., a pH of 12-14, a time of 4-8 h;
2) adding DEO as the crosslinking agent, etherifying at 20-30° C. for 24-36 h, controlling the mass ratio of DEO to HA and HPMC 1:5-3:1;
3) using hydrochloric acid to adjust pH to 5-6, concentrating in vacuum, and esterifying at 0.1 mPa in vacuum at 40-45° C. for 1-2 and
4) neutralizing and dehydrating with an ethanol solution (30-50% ethanol) containing 0.1-0.5% NaOH. dryint in vacuum at 0.08-0.09 mPa and 50-60° C. for 10-12 h, dissolving with phosphate buffer to obtain a mix solution at a pH of 6.9-7.6.

Two methods for synthesizing QAH are provided:

The first method for synthesizing QAH is using Oxidation, and the method comprises: dissolving Quaternary Ammonium Halide in water; mixing intensively with silver powder, adding hydrogen peroxide as an oxidant, and obtaining the solution of QAH.

HA and HPMC can be dissolved in the solution after filtering silve halide. The method is advantageous in simple, rapid, environmental, low consumption of materials, and not carrying in any impurities of metal ions and organic solvent, and the silver halide can be recovery and reuse. A chemical equation of the method is followed:

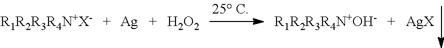

in which, X=Cl, Br; R₁, R₂, R₃, R₄ are four alphatic groups or aryl groups.

Preferably, Quaternary Ammonium in the method is tetrabutyl ammonium bromide (TBAB).

The second method for synthesizing QAH is using ethanol as a solvent, and the method comprises: dissolving, Quaternary Ammonium Halide and NaOH in ethanol, respectively; mixing the two kinds of solution rapidly; and obtaining a high-concentration solution of QAH after filtering sodium halide and eliminating the ethanol via vacuum concentration. A chemical equation of the method is followed:

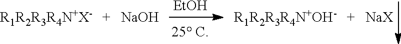

in which, X=Cl, Br; R1, R2, R3, R4 are four alphatic groups or aryl groups.

Preferably, Quaternary Ammonium in the method is trimethyloctyl ammonium chloride. (TMOAC).

In the invention, in order to prepare the composite gel of high crosslinking degree (20%-300%), QAH has been added as the catalyst of both alkali and phase transfer. Both of the solubility and utilization percentage of DEO are increased, which the solubility of DEO is more than 20% (mass fraction), and the utilization ratio of DEO is more than 90%.

Structure formulas of the two crosslinking agents are followed:

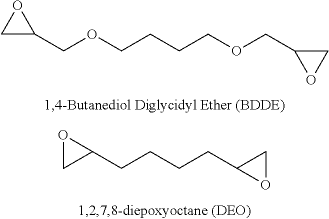 [SMILES: C(CCOCC1CO1)COCC1CO1 ; C(CCC1CO1)CC1CO1]

The chemical equation of the method is followed:

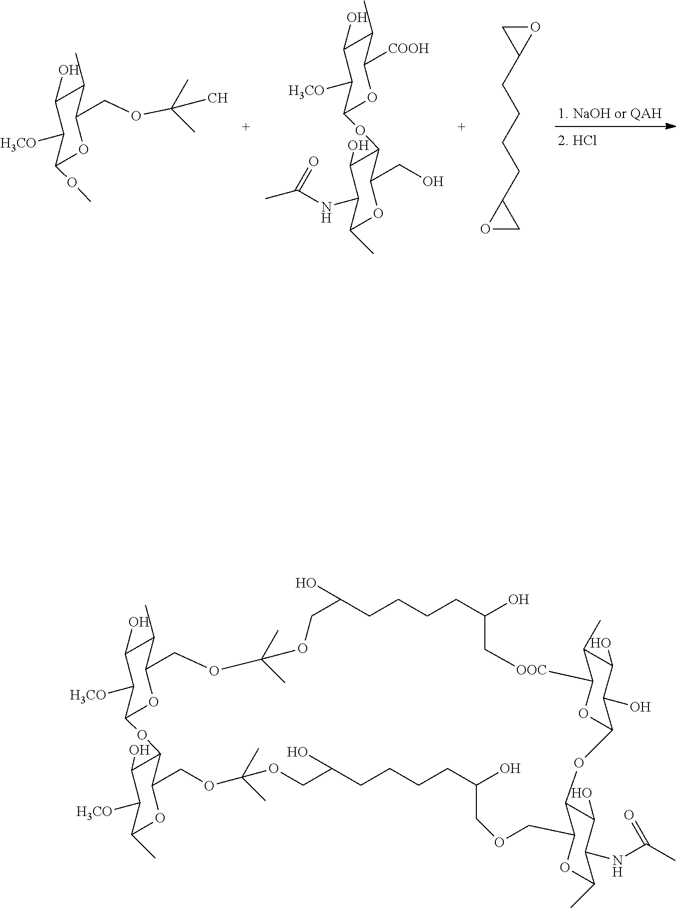

In the invention, the quaternary ammonium hydroxide is better than the composite of quaternary ammonium halide and NaOH. Because the ion intensity of the quaternary ammonium hydroxide is less than the composite of quaternary ammoniuin halide and NaOH, and the solubility of HA in water would be lower in the higher ion intensity. This lead to that the crosslinking reaction cannot proceed completely, and the utilization percentage of DEO would be lower. Therefore, the advantages of choosing quaternary ammonium hydroxide as catalyst of both alkali and phase transfer are high utilization percentage of DEO and that tbe crosslinking reaction can proceed completely.

In the invention, a method for eliminating the crosslinking agents DEO in the crosslinked gel of HA and HPMC are provided. DEO has been eliminated to a safe range of content by high pressure steam, thereby ensuring the safety of the products of composite gel.

The specific steps of the method for eliminating DE( )are followed:
1) adjusting, a pH of the composite gel to 7-7.5, rolling the composite gel by a rolling machine for 18-24 h to achieve a swelling equilibrium;
2) sealing the bottle with non-woven fabrics, a pore size of which is 0.1-0.2 μm, placing the bottle in an autoclave, and closing an air bleed valve;
3) when pressure is 0.12 mPa in vacuum and temperature is 105° C., opening the air bleed valve until pressure is 0.1 mPa in vacuum and temperature is 100° C., and closing the air bleed value: and
4) repeating step (c) for 4-6 times in 25-35 minutes, and then DEO can be eliminated in a safe range of content.

The residues of DEO in the composite gel can be determined by Gas Chromatography (GC). And the residues of DEO are lower than the detectable level of GC (2 μg/g or 2 ppm).

Advantages of the invention. are summarized as follows: in the invention, the composite gels have excellent properties of high thermal stability, acid and alkali resistance, hyaluronidase resistance and performance stability. The degradation rate of the composite gel is less than 1% in the condition of 125° C. for 0.5 h, and less than 10% in the condition of strong acid (pH=1) or strong alkali (pH=13) for 10 h, and only 2% in the hyaluronidase solution of 100 u/mL at 37° C. for 10 h.

### BRIEF DESCRIPTION OF THE DRAWINGS

The invention is described hereinbelow with reference to the accompanying drawings, in which:

FIG. 1 is an FTIR spectra between the composite gels (using DEO as crosslinking agent) in the invention and HA;

FIG. 2 is a ¹³C NMR spectra between the composite gels (using DEO as crosslinking agent) in the invention and HA;

FIG. 3 is the molecular changes of composite gel in vitro hyaluronidase hydrolysis (HAse 300 u/mL), determined by GPC;

FIG. 4 is the molecular changes of crosslinked HA in vitro hyaluronidase hydrolysis (HAse 300 u/mL), determined by GPC;

FIG. 5 is a gas chromatogram of DEO standard sample; and

FIG. 6 is a gas chromatogram of composite gel after high pressure steam.

### DETAILED DESCRIPTION OF THE EMBODIMENTS

### Example 1

### Preparation of QAH:

### Example 1-a

### Oxidation

A quaternary ammonium in the method is tetrabutyl ammonium bromide (TBAB). 6.44 g TBAB was dissolved in 100 mL distilled water to form a TBAB solution, 2.5 g silver powder were added into the TBAB solution and mixed at 25° C. 30% hydrogen peroxide were dropped slowly into the solution, the reaction was continued for 6 h. A silver nitrate solution was added into a supernatant of the reaction system after adding nitrate acid, and the oxidation reaction was complete if there was no silver bromide appeared. The solution of tetrabutyl ammonium hydroxide was obtained after filtering silver bromide, and HA was dissolved in the solution.

Example 1-b

### Ethanol as Solvent

A quaternary ammonium in the method is trimethyloctyl ammonium chloride (TmOAC).

4.18 g TMOAC was dissolved in 100 mL ethanol (90%), and 0.8 g NAOH was dissolved in 100 mL ethanol (90%), then the two solutions of ethanol were mixed quickly, and the reaction time was controlled at 12-18 h. The solution was vacuum-concentrated at 35-40° C. and 0.09 mPa in vacuum for 4-5 h after filtering NaCl. Then the high-concentration solution of QAH (80%-90%) was obtained, and the percentage content of ethanol was less than 5%.

Example 2

### Preparation of Composite Gel of HA and HPMC with DEO as Crosslinking Agent

The high-concentration solution of QAH in Example 1. was diluted to a content of 0.1 mol/L. Then 8 g HA (Bloomage Freda Biopharm Co., Ltd) and 2 g HPMC (Dow Chemical Company) were dissolved in the QAH solution for 12-14 h at 25° C., then 3 g DEO (J&K Scientific Ltd.,) was added into the reaction system for 24-26 h at 25° C. Thereafter, pH was adjusted to 4-5 with 2 mol/L hydrochloric acid, and water in the system was eliminated at 40° C. and 0.1 mPa in vacuum for 0.5-1 h. After that, the reaction system was neutralized and dehydrated using 200 mL NaOH solution (0.01% in 50% ethanol), and the composite gel was dried at 50-60° C. and 0.08-0.09 mPa in vacuum for 10 h, then dissolved with phosphate buffer (pH=7). A crosslinking degree of the composite gel was 30%.

### Example 2-a

### Determination of Swelling Degree

1 g composite gel after drying in vacuum was immersed in 200 ml phosphate buffer (pH=7) for 72 h to achieve the swelling equilibrium (the weight of the gel was constant). The free water at the surface of the gel was removed with filter paper, and the weight of the composite gel was 50 g. The swelling degree was 50:1.

### Example 2-b

### Hydrolysis in Vitro with Hyaluronidase

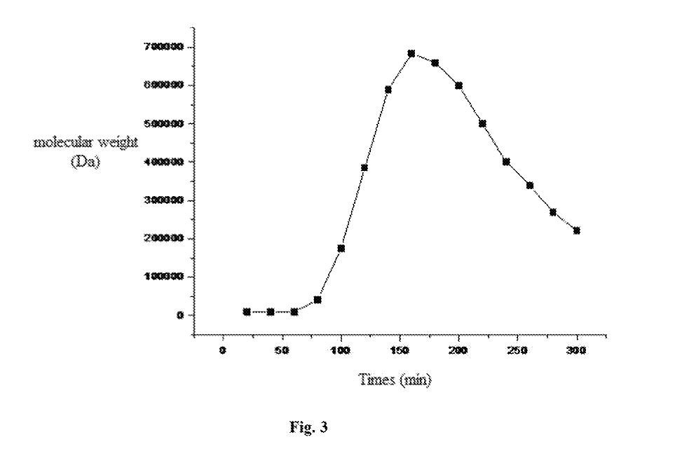

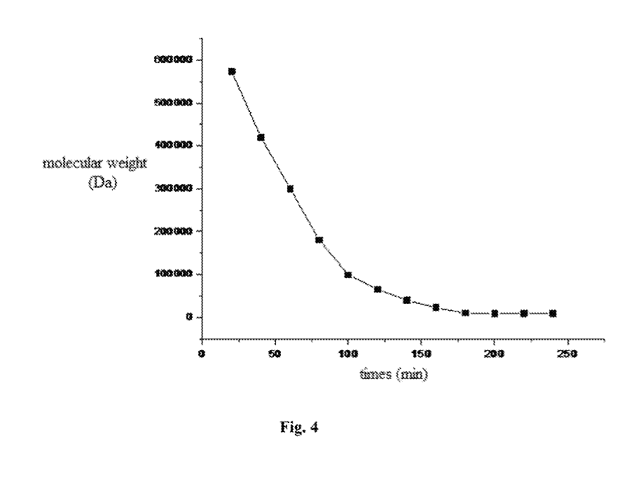

0.5 mL of 20 mg/mL (solid content) composite gel (Example 2) and crosslinked HA gel (Example 4) were added respectively into two colorimetric tubes, then 1500 unit of hyaluronidase and 2 mL distilled water were added for dilution, in an immersion oscillator registration at 37° C. 50 μL of the supernatant was cooled quickly to lower than 5° C. in ice-water bath every 20 minutes in 5 h. Then a molecular weight in each supernatant could be determined by GPC, and a hydrolysis would be complete until the molecular weight in the supernatant was constant. As shown in FIG. 3, the molecular weight of the composite gel was constant in the first 60 minutes, increased in the next 90 minutes and reached the peak, then decreased in the last 150 minutes, and the composite gel could not degrade completely in five hours; simultaneously, in FIG. 4, the molecular weight of the crosslinked HA gel decreased quickly, and the gel was degraded completely in 90 minutes. Therefore, a chemical stability of composite gel was better than that of the crosslinked HA gel.

### Example 2-c

### Test of Thermal Stability

5 g (accurate to 10 mg) of composite gel after swelling equilibrium in Example 2-a was collected, and 100 mL water was added to form a mixture. The mixture was placed in an oven at 80° C., for 24 h. Then the gel was weighed after cooling and drying, and the mass of the gel was 4.99 g, the degradation of the gel was less than 0.2%. Then another 5 g of composite gel was accurately weighed and placed in an autoclave sealed at 125° C. for 30 minutes. Then the gel was weighed, after cooling and drying, and the mass of the gel was 4.98 g, the degradation of the gel was less than 0.4%. If 5 g of the gel was placed in the autoclave sealed at 125° C. for 6 h, the mass of the gel was 3.78 g after cooling and drying free water in the surface of the gel, and the degradation of the gel was less than 25%. Therefore, the composite gel had high thermal stability.

### Example 2-d

### FTIR Spectra and Solid-¹³C NMR Spectra of Composite Gel with DEO as Crosslinking Agent

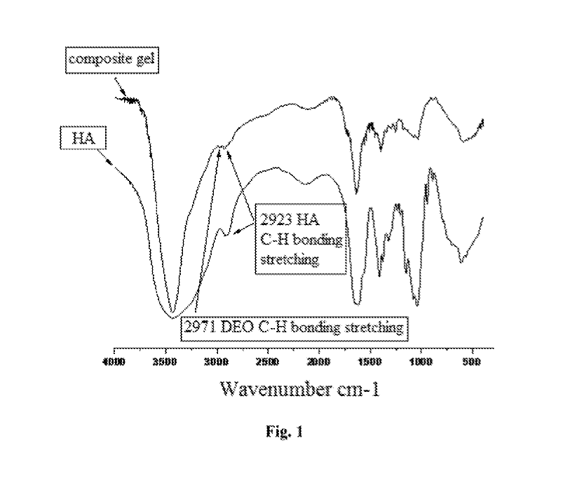

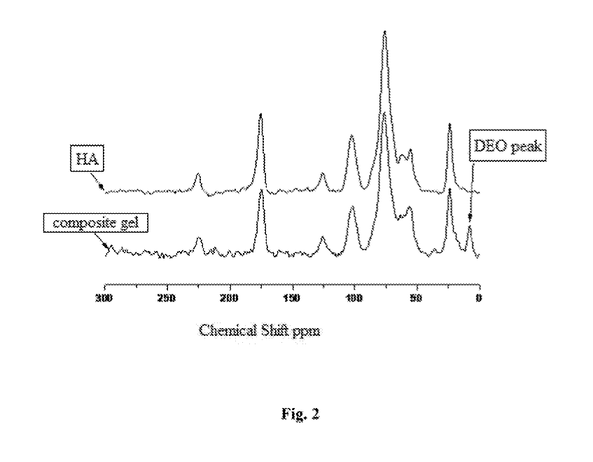

The composite gel powder which was prepared in example 2 was measured by FTIR and solid-¹³C NMR. As shown in FIG. 1, the peak near 2971 cm⁻¹ in the FT-IR spectra is distributed to the C—H bonding stretching of DEO; and as shown in FIG. 2, the peak near 8.05 ppm in the ¹³C NMR spectra is the characteristic peak of DEO.

### Example 2-e

### Elimination and Determination of DEO

1 g the dry composite gel which was prepared in example 2 is diluted to 20 mg/mL with PBS of pH=7 for 72 h to achieve swelling equilibrium. The bottle was sealed with non-woven fabrics whose pore size was 0.1-0.2 μm. The bottle was placed in an autoclave, then an air bleed valve was closed; when the pressure was 0.12 mPa in vacuum and the temperature was 105° C., the air bleed valve was opened until the pressure was 0.1 mPa in vacuum and the temperature was 100° C., and then the air bleed valve was closed. The last step was repeated for 4-6 times in 25-35 minutes so that DEO was decreased to a safe range of content.

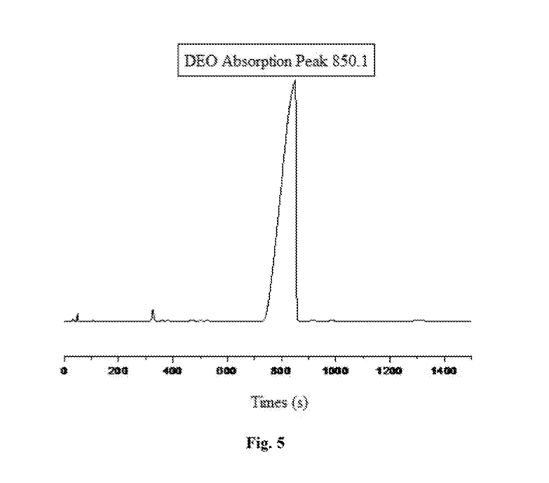

The standard sample was prepared by that 2 μL DEO was diluted with water in bottle for headspace-gas chromatography analysis. Then the bottle was sealed and put in the oven at 95° C. for 40 min. 1 mL of the headspace gas was collected and tested with gas chromatography, and a spectra, as shown in FIG. 5, was obtained.

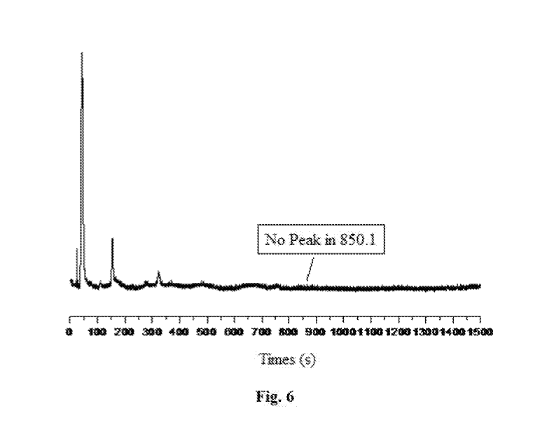

2 g (accurate to 0.1 mg) of composite gel was precisely weighed after high-pressure steam, and 8 mL water was added. Then the bottle was sealed and placed in the oven at 95° C. for 40 min, 1 mL of the headspace gas was collected and tested with gas chromatography, and a spectra, as shown in FIG. 6 was obtained.

According to FIG. 5 and FIG. 6, the residues of DEO in the composite gel were lower than the detectable level, 0.1 ppm. So that DEO was eliminated to a safe range of content.

### Example 3

### Preparation of Composite Gel of HA and HPMC with BDDE as Crosslinking Agent

8 g HA (Bloomage Freda Biopharm Co., Ltd) and 2 g HPMC. (Dow Chemical Company) are dissolved in 100 mL for 12-14 h at 25° C., then 1 g NaOH and 3 g BDDE (J&K Scientific Ltd.,) were added into the reaction system for 24-26 h at 25° C. pH was adjusted to 4-5 with 2 mol/L hydrochloric acid. Then, the reaction system was neutralised and dehydrated with 200 mL ethanol solution (50% in water). After that, the composite gel was dried at 50-60° C. and 0.08-0.09 mPa in vacuum for 10 h, and finally dissolved with phosphate buffer (pH=7).

### Contrast Sample

The high-concentration solution of QAH in Example 1 was diluted to the content of 0.1 mol/L. Then 10 g HA (Bloomage Freda Biopharm Co., Ltd) were dissolved in this QAH solution for 12-14 h at 25° C., then added 3 g DEO (J&K Scientific Ltd.,) into the reaction system for 24-26 h at 25° C. pH was adjusted to 4-5 with 2 mol/L hydrochloric acid, and water was eliminated from the system at 40° C. and 0.1 mPa in vacuum for 0.5-1 h. Thereafter, the reaction system was neutralized and dehydrated with 200 mL NaOH solution (0.01% in 50% ethanol). After that, the composite gel was dried at 50-60° C. and 0.08-0.09 mPa in vacuum for 10 h, and finally dissolved with phosphate buffer (pH=7). The crosslinking degree of the composite gel was 30%,

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

We claim:

1. A crosslinked gel composition of hyaluronic acid (HA) and hydroxypropyl methylcellulose (HPMC), the crosslinked gel composition being produced by using HA and HPMC as raw material and crosslinking with 1, 2, 7, 8-diepoxyoctane (DEO) or 1, 4-butanediol diglycidyl ether (BDDE), wherein the specific producing method for the crosslinked gel composition is:
proportionally mixing HA and HPMC solution;
after stirring thr reaction, adding sodium hydroxide solution into the mixed HA and HPMC solution until mixture is alkali;
when well mixed, adding DEO or BDDE into the mixture;
after stirring for reaction, adding acid until the mixture is week acidic;
solidifying and dewatering the mixture using acetone;
washing with organic solvent and then vacuum drying to produce HA-HPMC powder; and
repeatedly dissolving the powder to produce HA-HPMC gel composition.

2. The crosslinked gel composition of claim 1, wherein when producing highly crosslinked gel composition using DEO cross-linking agent quaternary ammonium hydroxide of tetrabutylammonium hydroxide or octyltrimethylammonium hydroxide is used as catalyst.

3. The crosslinked gel composition of claim 1, wherein a mass fraction of HPMC is in range of 1%˜50%.

4. The crosslinked gel composition of claim 1, wherein a mass ratio of HA monomer to di-epoxide cross-linking agent is 1:0.05˜3.

5. A method for producing the crosslinked gel compostion of claim 1, comprising.:
1) proportionally mixing HA and HPMC solution well and stirring for reaction for 16-18 hours;
2) adding sodium hydroxide solution until mixture is alkali and then adding DEO or BDDE into the mixture, stirring to mix thoroughly, and reacting for 24-36 hours under room temperature;
3) adding hydrochloric acid to adjust the pH to be 5-6, solidifying and dewatering the mixture using acetone, washing with ethyl alcohol absolute or acetone, and vacuum drying at 50° C. to produce HA-HPMC gel powder, and then redissolving the powder with neutral phosphate buffer solution to produce HA-HPMC gel composition.

6. A method for producing the crosslinked gel compostion of claim 1, comprising:
1) proportionally mixing tetrabutylammonium hydroxide or octyltrimethylammonium hydroxide solution, HPMC solution and HA powder well and stirring for 16-18 hours;
2) adding DEO crosslinking agent, stirring to mix thoroughly, and reacting for 18-36 hours;
3) adding hydrochloric acid to adjust the pH to be 5-6, vacuum concentrating for dehydration to make carboxyl group take part in cross-linking reaction, neutralizing the hydrochloric acid by ethanol solution with pH of 8-9 to dewater gel, and vacuum drying to produce HA-HPMC powder, and then repeatedly dissolving the powder with neutral phosphate buffer solution (PBS) to produce HA-HPMC gel composition.

7. The method of claim 6, wherein a mass ratio of HA to DEO is 1:0.2˜3.

8. The method of claim 6, wherein vacuum concentration is under 30-35° C., 0.1 MPa.

9. The method of claim 6, wherein the vacuum drying is carried out under condition of 50-60° C., 0.08-0.09 MPa, and is lasted for 10-12 hours.

10. The method of claim 6, wherein a mass concentration of the ethanol solution is in range of 25%-35%.

11. The method of claim 6, wherein a mass ratio of the quaternary ammonium hydroxide to the whole mixture is in range of 0.5-30%.

12. The method of claim 6, wherein a method for producing the tetrabutylammonium hydroxide is: well mixing elemental silver and tetrabutyl ammonium bromide aqueous solution, adding hydrogen peroxide as oxidizer to rapidly synthesize tetrabutylammonium hydroxide aqueous solution under room temperature, and filtering to remove silver bromide precipitation; 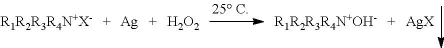
wherein, X═Br, and R1, R2, R3, R4 are normal-butyls.

13. The method of claim 6, wherein a method for producing octyltrimethylammonium hydroxide solution is a method for producing quaternary ammonium hydroxide by ethanol, the octyltrimethylammonium hydroxide solution is produced by firstly dissolving octyltrimethylammonium chloride and sodium hydroxide into 90% ethanol respectively, then mixing the two solutions and stirring, filtering to remove sodium chloride precipitation and obtain octyltrimethylammonium chloride alcoholic solution, and vacuum concentrating: 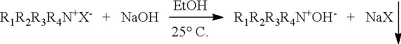
wherein, X═Cl, and R1, R2, R3 are methyls, R4 is a normal octyl.

14. A method for removing DEO cross-linking agent from HA and HPMC crosslinked gel composition produced by the method of claim 6, comprising:
1) adjusting pH value of the gel composition aqueous gel solution by acid to 7-8, rolling in a roller machine for 18-24 hours until HA is in water balance;
2) sealing lip of bottle with air-permeable and bacteria-impermeable paper, placing in an autoclave, closing a exhaust valve of the autoclave after the autoclave is heated to boil;
3) when pressure is increased to 0.12 MPa and temperature is increased to 105° C., opening the exhaust valve, and closing the exhaust valve when the pressure is decreased to 0.1 MPa and the temperature is decreased to 100° C.
4) repeating step 3) to increase the pressure and temperature, and so forth for 4-5 times to reach the aim of removing the DEO cross-linking agent from crosslinked HA.

15. The method of claim 14, wherein a concentration of the aqueous gel is 18-23 mg/mL.

16. The method of claim 14, wherein time for high pressure in steps 3 and 4 lasts for 28-32 minutes.
